(19) United States
(12) Patent Application Publication
(10) Pub. No.: US 2008/0153928 A1
(43) Pub. Date: Jun. 26, 2008

(54) Method for Providing and Analyzing an Animal Population Having an Essentially Identical Metabolome

(75) Inventors: Bennard van Ravenzwaay, Altrip (DE); Georgia Coelho Palermo Cunha, Limburgerhof (DE); Werner Mellert, Hassloch (DE); Ralf Looser, Berlin (DE); Tilmann B. Walk, Kleinmachnow (DE); Jan C. Wiemer, Berlin (DE); Volker Haake, Berlin (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/989,219

(22) PCT Filed: Jul. 10, 2006
(86) PCT No.: PCT/EP2006/064053

§ 371 Date: Jan. 22, 2008

(30) Foreign Application Priority Data
Jul. 25, 2005 (EP) 05016064.7
Aug. 26, 2005 (EP) 05018611.3

(51) Int. Cl. A61K 35/00; A01K 1/03

(52) U.S. Cl. 514/789; 119/417

(57) ABSTRACT

The present invention relates to a method for providing an animal population having an essentially identical metabolome comprising compiling an animal population being of essentially the same age, keeping said animal population for a time period sufficient for acclimatization under the following housing conditions: (i) constant temperature, (ii) constant humidity, (iii) physical separation of the animals of the animal population, (iv) feeding ad libitum, wherein the food to be fed is essentially free of chemical or microbial contaminance, (v) drinking libitum, wherein the drinking liquid is essentially free of chemical or microbial contaminance, (vi) constant illumination period, and providing the animal population after said time period. Further, the present invention relates to methods for the identification of a compound which effects the metabolome of an animal, or methods for the identification of a marker for such compounds. Moreover, the present invention encompasses methods for the identification of such compounds or markers thereof comprising metabolically analyzing a sample from at least one animal of an animal population.

The present invention relates to a method for providing an animal population having an essentially identical metabolome comprising compiling an animal population being of essentially the same age, keeping said animal population for a time period sufficient for acclimatization under the following housing conditions: (i) constant temperature, (ii) constant humidity, (iii) physical separation of the animals of the animal population, (iv) feeding ad libitum, wherein the food to be fed is essentially free of chemical or microbial contaminants, (v) drinking liquid ad libitum, wherein the drinking liquid is essentially free of chemical or microbial contaminants, (vi) constant illumination period, and providing the animal population after said time period. Further, the present invention relates to methods for the identification of a compound which effects the metabolome of an animal, or methods for the identification of a marker for such compounds. Moreover, the present invention encompasses methods for the identification of such compounds or markers thereof comprising metabolically analyzing a sample from at least one animal of an animal population.

State-of-the-art techniques of phenotype analysis of organisms comprise, inter alia, analysis of the entire genome of said organism, called genomics, analysis of the entirety of the proteins, called proteomics, and the analysis of the entirety of RNA transcripts. More recently, these fundamental techniques of phenotypic analysis have been completed by the analysis of the metabolome, the entirety of metabolites, of an organism. This analysis is called metabolomics or, sometimes, metabonomics. Metabolomics can be defined as the qualitative and quantitative determination of all low molecular weight compounds (i.e. metabolites) in an organism's cell or body fluid at a specific time and under specific environmental conditions. An advantage of metabolomics is that effects caused by exogenous factors can be immediately monitored due to metabolic changes which usually appear much earlier than changes, if any, in the proteome or even in the genome.

Various techniques have been described already for the analysis of the metabolome of an organism. These techniques include, for instance, mass spectroscopy, NMR, Fourier transform infrared (FT-IR) spectroscopy, and flame ionisation detection (FID), optionally coupled to chromatographic separation techniques such as liquid chromatography, gas chromatography or HPLC. These techniques allow high-throughput screening of large populations of organisms for variations in the composition of their metabolome, i.e. they allow to determine a metabolic phenotype. An organism's metabolic phenotype is the entirety of its metabolites (metabolome) at a certain time and is the result of complex interactions of its genetic composition and the living environment of the organism. Accordingly, differences in the metabolome of the individuals of a population of organisms may be caused not only by differences in the genome but also by environmental factors which influence metabolic activities. Metabolomics, thus, allows to determine even effects of exogenous factors which do not influence the genome, transcription or proteome of an organism immediately. For instance, a toxic compound is harmful for an organism but must not necessarily cause changes in the genome of said organism.

At present, a drawback in metabolomics in comparison to other principle phenotypic analysis approvals is that organisms and, in particular, animals which are used in metabolic studies in the prior art do not share a common metabolome at the beginning of a study. In genomics, for instance, a population having an essentially identical genome can be easily provided by state-of-the-art cloning techniques. However, it would be highly desirable to detect metabolic influences of exogenous factors, such as toxic compounds or drugs under optimized conditions. Techniques for establishing animals having an essentially identical metabolome are the basis for a reliable and efficient metabolome analysis. Notwithstanding the need therefore, such techniques are not yet described.

Accordingly, the technical problem underlying the present invention must be seen as the provision of methods for complying with the aforementioned needs, i.e. providing an animal population which has an essentially identical metabolome suitable for reliable metabolome studies and reliable analysis. The technical problem is solved by the embodiments characterized in the claims and described herein below.

Accordingly, the present invention relates to a method for providing an animal population having an essentially identical metabolome comprising:
a) compiling an animal population being of essentially the same age;
b) keeping said animal population of step a) for a first time period sufficient for acclimatization under the following housing conditions:
   i) constant temperature;
   ii) constant humidity;
   iii) physical separation of the animals of the animal population;
   iv) feeding ad libitum, wherein the food to be fed is essentially free of chemical or microbial contaminants;
   v) drinking ad libitum, wherein the drinking liquid is essentially free of chemical or microbial contaminants;
   vi) constant illumination period; and
c) providing the animal population of step b) after said first time period.

The term “method for providing” as used herein does, preferably, not encompass methods of treatment of the animal body. Specifically, the method referred to herein is neither suitable for medical treatment or therapy of any disease or disorder nor suitable to improve or maintain the general well-being of the animals of the animal population in comparison to animals kept under other physiological conditions. Moreover, the term does not include any breeding techniques per se.

The term “animal population” relates to a plurality of animals. A plurality of animals as used herein is a group of animals consisting of at least two, preferably, 5 to 120, more preferably 5 to 25 of said animals per sex, dose and time point. The animals of the animal population are of the same species and, preferably, of the same strain. Preferred animals to be used in the method of the present invention are mammals, more preferably rodents, most preferably, rats or mice. If rats are used for the method of the present invention, R is furthermore preferred that these rats are wistar (CrlGlxBrlHan:Wi) rats (Charles River, USA). Further preferred rat strains are: BDIX strains; BDIX/CrCrl, BDIX/OrlCrl, Brown Norway strains; BN/CrlCrlj, BN/Crl, BN/OrlCrl, BN/OrlCrl, BN/SsNHsdMcwiCrl, Buffalo strain; BUF/CrCrl; Fischer strains, F344/DuCrl, F344/DuCrlCrlj, F344/IcoCrl, F344/DuCrl, SASCO Fischer strain, F344/NCrl; Copenhagen strains, COP/CrCrl, COP/NCrl; Cotton strain, COT/NCrl; Dahl/SS strain, SS/JrHsdMcwiCrl; Fawn Hooded strain, FHH/EurMcwiCrl; GK strain, GK/Crlj; Lewis strains, LEW/CrlCrl; LEW/Crl; Noble strain, NBL/CrlCrl; SHR strains, SHR/NCrlCrlj, SHR/NCrl, SHR/NCrl; WAG strain, WAG/RijCrl; Wistar Furth strains, WF/CrCrl, WF/IcoCrl; WKY strains, WKY/NCrl, WKY/NCrlCrlj, WKY/NIcoCrl; ZDF strains, ZDF/Crl-Lepr^{fa}, ZDF/Crl-Lepr^{fa}; CD strains, Crl:CD(SD), Crlj:CD(SD), Crl:CD(SD), Crl:CD(SD); SASCO SD strain, Crl:SD; OFA strains, Crl:OFA(SD), Crl:OFA(SD)-hr; DIO strain, Crl:CD(SD)DR; DR strain, Crl:CD(SD)DR; Donryu strain, Crlj:DON; LEC strain, Crlj:LEC; Wistar strains, Crlj:WI, Crl:WI; Wistar Han strains, Crl:WI(Han), Crl:WI(Han), Crl:WI (Han), Crl:WI(Han); Wistar WU strain, Crl:WI(WU) or any strain which is derived from the aforementioned strains by way of cross breeding or genetic manipulation. The aforementioned strains are well known in the art and commercially available, e.g., via Charles River, USA or Harlan, USA. If mice are used for the method of the present invention, it is preferred that the mice are C57BL/GNCrl mice (Charles River, USA). Other preferred mouse strains are: CD-1 strains, Crlj:CD1(ICR), Crl:CD1(ICR), Crl:CD1(ICR), Crl:CD1(ICR), Crl:CD1(ICR); CF-1 strains, Crl:CF1, Crl:OF1; CFW strains, Crl:CFW(SW); NMRI strains, Crl:NMRI, Crl:NMRI (Han); PGP strain, Crl:CF1-Abcb1a; SKH1 strains, Crl:SKH1-hr, Crl:SKH1-hr, SKH2 strain, Crl:SKH2-hr, A/J strain, A/J; 129 strains, 129S2/SvPasCrl, 129S2/SvPasCrl; 129/S1/Sv-p⁺Tyr⁺Kit^{SI-J}/Crl, 129/s1/svlmJ; AKR strain, AKR/NCrl; BALB/c strains, BALB/cAnNCrlCrlj, BALB/CAnNCrl, BALB/cByJ; C3H strains, C3H/HeJCrl, C3H/HeNCrlCrlj, C3H/HeNcrl, C3H/HeOuJ; C57BL/6 strains, C57BL/6JCrl, C57BL/6J, C57BL/6JCrl, C57BL/6NCrlCrlj, C57BL/6NCrl, C57BL/6J, C57BL/6JCrl, C57BL/6J; C57BL/10 strains, C57BL/10JCrl, C57BL/10JCrl; CBA strains, CBA/CaCrl, CBA/J, CBA/JNCrlj, CBA/JNCrljCrlg; CB17 strain, CB17/IcrCrl; DBA/1 strains, DBA/1JCRL, DBA/1JNCrlj, DBA/1NIcoCrl; DBA/2 strains, DBA/2J, DBA/2NCrlCrlj, DBA/2NCrl, DBA/2JCrl; FVB strain, FVB/NCrl; NC strain, NC/NgaTndCrlj; NOD strain, NOD/LtJCrl; SJL strains, SJL/JOrlCrl, SJL/JOrl/CrlCrlj, SJL/J, SJL/JCrl; B6C3F1 strains, B6C3F1/Crl, B6C3F1/Crlj; B6CBAF1 strains, B6CBAF1/Crl, B6CBAF1/J, B6CBAF1/Crl; BDF1 (B6D2F1) strains, B6D2F1/Crl, B6D2F1/J, B6D2F1/Crlj; B6SJLF1 strain, B6SJLF1/J; C3D2F1 strain, C3D2F1/Crl; CDF1 (CD2F1) strains, CD2F1/Crl, CD2F1/Crlj, CD2F1/Crl; CBAB6F1 strain, CBAB6F1/Crl; CB6F1 strain, CB6F1/Crl; NMRCF1 strain, NMRCF1/Crl or any strain which is derived from the aforementioned strains by way of cross breeding or genetic manipulation. The aforementioned strains are well known in the art and commercially available, e.g., via Charles River, USA or Harlan, USA. Further preferred animals are dogs. Preferred dogs encompass beagles, more preferably, the beagle strains HsdRdg:DOBE or HsdHFr:DOBE or strains which are derived from the aforementioned strains by way of cross breeding or genetic manipulation. The aforementioned strains can be purchased from Harlan, USA. Other preferred beagles are from an inbreed strain and can be purchased from BASF AG, Germany. However, the animals to be used are not limited to the animals mentioned before and may further be selected from the group consisting of: cats, horses, cows, sheep, goats, rabbits, fishes, birds, and insects, such as fruitflys (drosophila).

The term “metabolome” as used herein refers to the entirety of metabolites in a cell, tissue, organ or entire animal. The metabolites are, preferably, small molecule compounds, such as substrates for enzymes of metabolic pathways, intermediates of such pathways or the products obtained by a metabolic pathway. Metabolic pathways are well known in the art and may vary between species. Preferably, said pathways include at least citric acid cycle, respiratory chain, photosynthesis, photorespiration, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including e.g. flavonoids and isoflavonoids), isoprenoids (including e.g. terpenes, sterols, steroids, carotenoids, xanthophylls), carbohydrates, phenylpropanoids and derivatives, alkaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal all function, organ function or animal growth, development or health. Moreover, small molecule metabolites further comprise secondary metabolites having essential ecological function, e.g. metabolites which allow an organism to adapt to its environment. Furthermore, metabolites are not limited to said primary and secondary metabolites and further encompass artificial small molecule compounds. Said artificial small molecule compounds are derived from exogenously provided small molecules which are administered or taken up by an organism but are not primary or secondary metabolites as defined above. For instance, artificial small molecule compounds may be metabolic products obtained from drugs by metabolic pathways of the animal. Moreover, metabolites further include peptides, oligopeptides, polypeptides, oligonucleotides and polynucleotides, such as RNA or DNA. More preferably, a metabolite has a molecular weight of 50 Da (Dalton) to 30,000 Da, most preferably less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. Preferably, a metabolite has, however, a molecular weight of at least 50 Da. Most preferably, a metabolite in accordance with the present invention has a molecular weight of 50 Da up to 1,500 Da.

The term “essentially identical metabolome” means that all individuals of the animal population provided by the method of the present invention have synchronized metabolic activities resulting (i) in the presence of essentially the same metabolites in the metabolome of each individual of the population and (ii) in amounts of said metabolites which are essentially identical for each of the individuals of the animal population. Preferably, synchronized metabolic activities as used herein means that all metabolic pathways which affect the metabolome of the animals are active in essentially the same cells, tissues or organs at essentially the same time, gene expression levels are in all animals are essentially identical and artificial small molecules are available for all animals in essentially identical amounts. It is to be understood that the metabolite amounts may vary between the individuals of the animal population within certain limits. Whether the same metabolites are present in essentially the same amounts in the individuals of the animal population referred to in accordance with the present invention, can be determined by various qualitative and/or quantitative compound analysis techniques. These techniques include but are not limited to chromatographic techniques for compound separation coupled to compound analysis techniques such as mass spectrometry (MS), Fourier transform ion resonance (FT-IR) spectroscopy, or FiD. Preferred in accordance with the present invention is a quantitative and/or qualitative determination of the metabolites by using liquid- and gas-chromatography coupled mass spectrometry (LC-MS and GC-MS). Details of said preferred methods are described below. Preferably, an animal population has an essentially identical metabolome if the mass spectra generated by one of these techniques are essentially identical for each individual of the population. Said mass spectra are essentially identical if all major peaks which are detectable by, e.g., a commercial peak annotation algorithm, such as ChemStation (Agilent Technologies, USA), Analyst (MDS SCIEX, Canada) or AMDIS (NIST, USA), appear in all spectra at essentially identical chromatographic retention times. As discussed above, minor variations are tolerable if they do not result in statistically significant differences. Whether a variation is minor in accordance with the present invention may be determined by suitable statistical algorithms well known to the person skilled in the art such as principal component analysis (PCA) or partial least square tests (PLS). Preferably, an essentially identical metabolome of the animals of the animal population can be determined together in a multivariate analysis (e.g., PCA) or hierarchical clustering.

The term “compiling” as used herein refers to selecting the animals from any source to establish the animal population to be subjected to the method of the present invention. Accordingly, the animals may be progeny of the same mother animal or progeny of different mother animals. In case a single progeny of one mother animal is used as a source, either the entire progeny may be used for compiling the animal population or selected animals of the progeny may be used. Compiling as used herein is carried out with respect to the age of the animals, i.e. all individuals of the population shall have essentially the same age as described below in detail. However, further characteristics may be taken into account. In addition, such as weight, sex, overall appearance (e.g. only healthy animal by appearance may be selected).

The term “essentially the same age” means that the animals have a comparable status of development, e.g. the animals may be embryos, juveniles or adults. A preferred age of the animals to be used in the method of the present invention is an age of the adolescence stage, preferably young adolescence stage. The animals of the animal population, preferably, have an age with the range of X in day, wherein X is the envisaged age of the animal population and n is selected from an integer of 1 to 5 days, and, more preferably, n is 1 day. In other words, a given animal of the population shall be at most one day older or younger than the average age of the animals of the animal population. Most preferably, all animals of the population are of age X. Such animals can be provided by compiling animals which are progeny of one litter, i.e. littermates, or which are compiled from different litters from the same day. In case embryos are to be used, it is to be understood that essentially the same age relates to their developmental stages. The developmental stages of embryos from various species can be determined by techniques well known in the art. They may be calculated, e.g., based on the time point of fertilization. Moreover, individual embryos can be developmentally staged due to known morphological features. Moreover, in case embryos are used, it is further to be understood that the pregnant mothers carrying said embryos shall be kept under the conditions referred to herein.

If rats or mice are used as animals in the method of the present invention, it is preferred that the animals are of age X±1 day, wherein X is selected from an integer of 10 to 100 days, more preferably, an integer of 20 to 80 days, and, most preferably, X is 63, 64 or 65 days after birth. Most preferably, X is 64 days after birth. If dogs are used as animals in the method of the present invention, X is preferably 6 months after birth.

The term “keeping” as used in accordance with the method of the present invention refers to particular housing, feeding, drinking and environmental conditions which are applied to the animals of the animal population. It is preferred that the animals are kept under conditions as set forth in the OECD Guideline For The Testing Of Chemicals No: 407. Moreover, particular conditions are described as follows.
i) All animals of the animal population are kept under the same constant temperature. Care should be taken to choose a temperature for carrying out the method of the present invention which does not stress the animals. Preferably, temperature should be 20 to 24±2° C., more preferably 22±2° C., most preferably 22, 23 or 24° C.
ii) Moreover, all animals of the animal population are kept under the same constant humidity. The humidity should be at least 30%, but should not exceed 70%. However, in rare exceptional situations (such as during room or cage clearing) humidity may even exceed 70%. Preferably, humidity is 50-60%.
iii) Physical separation of the animals of the animal population has been found to be also important for the method of the present invention. Accordingly, each animal of the animal population must be kept in a separate space, e.g. a separate cage.
iv) The animals of the animal population are fed ad libitum. The food to be used must be essentially free of chemical or microbial contaminants. The standards to be applied are laid down in Fed. Reg. Vol. 44, No. 91, May 9, 1979, p. 27354. Most preferably, microbial contaminants such as bacteria are below 5×10⁵ cells per g of food. Such food may be purchased from Provimi Kliba SA Kaiseraugst (Switzerland) as Ground Kliba mouse/rat maintenance diet “GLP” meal.
v) The animals of the animal population are supplied ad libitum with a drinking liquid. Preferably, said liquid is water. However, other liquids on water basis may be used as well. Such liquids may comprise, for instance, nutritions, vitamins or minerals which are required by the animals. If water is used as drinking liquid, the water shall be free of chemical and microbial contaminants as laid down in the European Drinking Water Directive 981831EG.
vi) Finally, each animal of the animal population must be subjected to the same constant illumination periods. Constant illumination is achieved, preferably, by artificial lightning (normal solar spectrum). The illumination period is 12 hours light followed by 12 hours darkness. Then the illumination period starts again. A preferred illumination period, thus, is 12 hours light, from 6:00 to 18:00, and 12 hours darkness, from 18:00 to 6:00.

The aforementioned housing conditions can be applied to the animals by using a common storage space for the cages comprising the physically separated animals. Said common storage space may be an animal room or house. By keeping all animals of the population in the same room, constant humidity, temperature and illumination period can be easily achieved by regulating these parameters for the entire room or house. Regulation of the parameters is preferably assisted by automation and the parameters are constantly monitored.

Under the term “first time period sufficient for acclimatization” it is to be understood that the animals of the animal population must be kept under the aforementioned particular housing conditions for a time period which allows synchronization of the metabolic activities of the animals so that the animals are acclimatized and have essentially the same metabolome. Specifically, the said first time period shall be of sufficient length as to allow all individuals of the population to adopt the same circadian rhythm, food digestion rhythm, or quiescence/movement periods. Moreover, the first time period shall allow each animal to adjust its biochemical and physiological parameters in response to the applied environmental conditions, such as humidity and temperature. Preferably, said first time period has a length of 5 to 10 days, more preferably 6 to 8, and most preferably 7 days.

In the studies underlying the present invention, it has been surprisingly found that an animal population having essentially the same metabolome can be provided by the method of the present invention. The superior results achieved by said method depend, however, on strictly obeying the aforementioned housing conditions and the criteria for compiling the animal population, i.e. compiling with respect to the age of the animals of the animal population. The later issue is particular surprising because according to the OECD Guideline No: 407, loc. cit., care should be taken with respect to the weight or sex when selecting or compiling animals for analytical purposes. Advantageously, by using the method of the present invention, it is possible to generate an animal population which can be applied for comparative metabolomics. Because of the essentially identical metabolome it is possible to reliably and efficiently study, e.g., toxic effects of compounds or to determine modes of action of compounds such as drugs or drug candidates. Moreover, the method of the present invention can be easily implemented in already existing animal facilities and is, thus, cost effective.

The present invention also encompasses a method for the identification of a compound which effects the metabolome of an animal comprising:
a) providing an animal population using the steps of the method of claim 1;
b) administering to said animal population a compound suspected to effect the metabolome of an animal; and
c) analyzing the metabolome of the animal population of step b).

The term “identification” as used herein means that the method of the present invention is to be applied for identifying or screening compounds which effect the metabolome of an animal. Accordingly, it is envisaged that the method will provide data which allow to identify a compound which effects the metabolome of an animal. Such data may be obtained by various techniques for analyzing the metabolome of an animal described in detail below. Data may be in the form of raw data or processed data. Preferably, the result of the metabolome analysis in form of raw data or processed data will be compared with corresponding data obtained from a reference. Accordingly, it is to be understood that identification as used in accordance with the method of the present invention may require further steps. However, these further steps relate to well known techniques in comparative analysis including those mentioned before and below. Moreover, identification as used herein encompasses preferably identification of the compounds' property to elicit an effect to the metabolome of an animal, in principle, regardless of the kind of the effect or mode of action. Moreover, the term further encompasses, preferably, the identification of the kind of effect which is elicited or event the precise mode of action. Therefore, the method of the present invention may be also used for identifying a particular metabolic pathway influenced by a compound or a certain mode of action of a compound. More preferably, the method for identification described above and below may be used to

The term “compound which effects the metabolome of an animal” encompasses all classes of chemical compounds. Preferably, a compound as used herein is a small molecule compound, a peptide, a polypeptide or a polynucleotide. Small molecules as referred to in accordance with the present invention include inorganic and organic molecules having a low molecular weight, preferably a molecular weight which is lower than 30,000 kDa, more preferably lower than 20,000 Da, 10,000 Da, 8,000 Da, 5,000 Da, 3,000 Da, 2,000 Da, 1,000 Da or 500 Da. Most preferably, said compound is either suspected to be a toxic compound, a nutrient, a nutraceutical or a therapeutically active compound (i.e. a drug). A drug in accordance with the present invention will have a therapeutic effect on the animal, i.e. it will treat or ameliorate a medical condition. Said medical condition may be a disease or disorder or an impaired well-being of an animal. Amelioration or treatment can be monitored by the appearance or degree of the symptoms of the said disease, disorder or impairment. Drugs in accordance with the present invention also include drug precursors which are converted in vivo into the therapeutically active drug and compounds suspected to be drugs, i.e. drug candidates. Peptides or polypeptides to be used in accordance with the method of the present invention indude naturally occurring peptides and polypeptides as well as artificial peptides and polypeptides. Naturally occurring peptides and polypeptides can be obtained from all kinds of organisms including plants, animals, fungi, bacteria or viruses. Artificial peptides or polypeptides can be generated by random peptide synthesis techniques, for instance. Preferred peptides or polypeptides to be used in accordance with the present invention are those which directly or indirectly influence metabolic activities or metabolic pathways in an animal according to the present invention. More preferably, said polypeptides or peptides have a therapeutic value, such as peptide hormones, growth factors, survival factors, cytokines, receptors for said polypeptides, antibodies or biologically active fragments thereof. Polynucleotides to be used in accordance with the method of the present invention are RNA or DNA molecules. Preferably, said polynucleotides encode peptides or polypeptides which directly or indirectly influence metabolic activities or metabolic pathways. For instance, suitable polynucleotides may encode enzymes, preferably those of metabolic pathways, peptides or polypeptides as specified above or peptides or polypeptides which regulate the expression of the aforementioned peptides or polypeptides, such as transcription factors. Moreover, polynucleotides to be used in the method of the present invention as compounds may be polynucleotides which interfere with gene expression (i.e. transcription or translation), such as anti-sense RNA molecules, anti-sense oligonucleotides or small interfering RNA (siRNA) to be used for RNA interference technology (RNAi).

The compounds referred to in accordance with the present invention are screened for their capability to effect the metabolome of an animal. Thus, a compound in the sense of the present invention will alter the composition of the metabolome of an animal upon administration. Said effect to the metabolome of an animal may be either a qualitative or quantitative effect. A qualitative effect to the metabolome as used herein means that at least one metabolite of the metabolome of the animal is absent or at least one additional metabolite is present after administration of the compound. A quantitative effect as used in accordance with the present invention means that the amount of at least one metabolite is altered, i.e. higher or lower, after administration of said compound. It is to be understood that a compound which effects the metabolome of an animal may also influence the biological function of the cells, the tissues or the organs of said animal and may cause intoxications, health improvements, health impairments or disorders.

The term “administering” as used herein includes all techniques by which the compounds may be provided systemically to the animal. Moreover, the term encompasses techniques for delivering the compounds to the suspected site of action such as a potential target tissue or organ, i.e. topical administration. The compounds to be administered in accordance with the present invention may be comprised in a composition further comprising suitable carriers, such as pharmaceutical carriers, excipients and/or diluents. Examples for well known diluents include phosphate-buffered saline solutions, water, emulsions, such as oil/water emulsions, various types of wetting agents, sterile solutions etc. Administration of the compounds or the aforementioned suitable compositions may be effected by different ways, e.g. by intravenous, intraperitoneal, subcutaneous, intramuscular, topical, intradermal, intranasal or intrabronchial administration. Preferably, administration is achieved by oral administration, most preferably the compound is admixed to the drinking liquid or the food. Suitable treatment and dosage regimens are well known to the person skilled in the art and a preferred treatment and dosage regimen is described in the Examples. Preferably, the animals of the test animal population (i.e. the animal population to which the compound is to be administered) are subdivided into at least one group of males and at least one group of females and at least one high and at least one low dose group.

The term “analyzing the metabolome” as used in accordance with the present invention refers to techniques for quantitatively or qualitatively analyzing the metabolome. In a first step, said qualitatively or quantitatively analyzing the metabolome comprises determining qualitatively and qualitatively the composition of the metabolome, i.e. the metabolites. Means and methods for qualitatively analyzing the metabolome comprise those which are capable to determine the presence or absence of the metabolites comprised by the metabolome. Means and methods for quantitatively analyzing the metabolome are those which determine the amount of the said metabolites. It is to be understood that there are means and methods which may determine the presence or absence as well as the amount of the metabolites and, thus, allow a combination of qualitative and quantitative analysis. Moreover, in accordance with the methods of the present invention it is not necessarily required to determine the entire metabolites of the metabolome. Rather, analysis of the metabolome may be carried out by determining the presence or absence or the amount of a portion of the metabolites found to be characteristic, a predetermined set of metabolites or a metabolic profile for the metabolome. Characteristic or predetermined metabolites comprise known metabolites as well as so called known unknowns. The later ones are metabolites which are merely known from their signal in, e.g., a mass spectra. The chemical nature of said known unknowns, however, is not precisely known. A metabolic profile as used herein relates to every kind of unique identifier for a certain metabolome, i.e. a fingerprint of a metabolome. Qualitative or quantitative analysis of the metabolome is, preferably, carried out by using compound analysis techniques. Suitable devices for such determination of compounds are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen, Journal of Chromatography A, 703, 1995: 37-57, U.S. Pat. No. 4,540,884 or U.S. Pat. No. 5,397,894, the disclosure content of which is hereby incorporated by reference. As an alternative or in addition to mass spectrometry techniques the following techniques may be used for compound determination: nuclear magnetic resonance (NMR), magnetic resonance imaging (MRI), Fourier transform infrared analysis (FT-IR), ultra violet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado.

In a further step, analyzing the metabolome as used herein, preferably, includes a comparison of the metabolites, amounts of metabolites or the metabolic profile to a corresponding reference. Said reference may be derived from a metabolome analysis of an animal population to which no compounds suspected to effect the metabolome of an animal has been administered, whereby it is preferred that this reference animal population has been treated in an otherwise identical manner. Further suitable references for a comparison in accordance with the present invention may be derived from a metabolome analysis of animals to which compounds have been administered which are known to effect the metabolome. Such compounds may be compounds which are toxic or which are therapeutically active via a known mode of action. Thus, by using animals treated with the aforementioned compounds known to effect the metabolome as reference, it may not only be determined whether a new compound has an effect to the metabolome in general. Rather, it may be further determined whether said new compound elicits toxic effects or may be therapeutically active via a certain mode of action or has at least the potential to do so. Preferably, the resulting data of a metabolome analysis of the references described before are stored and provided in form of a suitable database. Moreover, data of a metabolome analysis carried out by the method of the present invention for new compounds may also serve as reference for other new compounds and may be stored and provided also by the said data base for further analysis. Comparing as used herein encompasses comparison of the raw data obtained by the analysis of the metabolome or any kind of processed data derived from said raw data. Suitable means and methods for data comparison are well known in the art and include, for instance, principal component analysis (PCA) or partially least square tests (PLS). In principle, any statistical test which allows to determine whether metabolites, amounts thereof or a metabolic profile as described above differs significantly between different animals or time points of determination is suitable for carrying out the comparison referred to herein. The aforementioned differences can, in principle, be determined by pattern recognition algorithms, statistical test algorithms and/or multivariate algorithms, e.g., Principal Component Analysis (PCA), Simple Component Analysis (SCA), Independent Component Analysis (ICA), Principal Component Regression (PCR), Partial Least Squares (PLS), PLS Discriminant Analysis (PLS-DA), Support Vector Machines (SVM), Neural Networks, Bayesian Networks, Bayesian Learning Networks, Mutual Information, Backpropagation Networks, symmetrical Feed-Forward Networks, Self-Organizing Maps (SOMs), Genetic Algorithms, Hierarchical or K-Mean Clustering, Anova, Student's t-Test, Kruskal-Wallis Test, Mann-Whitney Test, Tukey-Kramer Test or Hsu's Best Test. Data processing as referred to above may preferably include a data validation step. In said data validation step, inconsistent data are excluded from further analysis. Inconsistent data may result technical problems during the qualitative or quantitative determination of the composition of the metabolome. Therefore, it is envisaged that the technical parameters of the devices used for determination are constantly monitored and provided for data validation in a suitable database. Further validation of the data may be achieved by internal validation tools which statistically evaluate each measured value or parameter of a data set. For example, if mass spectrometry is used for analyzing the metabolome, a raw data set comprising peaks is generated. Said peaks appear at a certain position in a three-dimensional space consisting of a retention time range, an intensity range and a mass-to-charge (m/z) ratio range. Each of said peaks may be evaluated and confirmed by peak validation algorithms such as ChemStation (Agilent Technologies, USA), Analyst (MDS SCIEX, Canada) or AMDIS (NIST, USA).

Furthermore, animal- or housing-related data may be taken into account for data validation. For example, if the housing conditions vary, the metabolome of an animal of the animal population may be adversely influenced. Such an adversely influenced metabolome of an animal may, however, cause false positive or negative results. Therefore, it is preferred to also monitor animal- or housing-related data as described below in detail and to regard these data during data validation.

Moreover, data processing, preferably, includes normalization of the raw data with respect to internal references. For example, peaks which can be allocated to known metabolites may be used to normalize peaks for unknown metabolites within a data set obtained for a metabolome of an individual of the animal population with respect to, e.g., signal intensity.

Moreover, data processing may be further required to create coherent data by converting data in a numeric format, converting the data into a common unit format and/or dimensionally reducing the data. The data may be further integrated in that information relating to the sample or animal is allocated thereto. Suitable means and methods for creating such coherent data are disclosed in WO 03/046798, the disclosure of which is hereby incorporated by reference.

The data processing steps and the comparison referred to above are preferably assisted by automation, e.g., by a suitable computer program which runs on a computer device. The computer device is, preferably, operatively linked to the various data bases referred to in accordance with the present invention. Accordingly, in one embodiment, the present invention also relates to a database or system of databases which comprise the results obtained by the method of the present invention. More preferably, such a database or system of databases comprises information relating to the metabolomes elicited by compounds which are suspect to affect the metabolome of an organism, such as toxic or therapeutically active compounds (i.e. drugs). Thus, the database may be used as a tool (reference database) for screening assays which aim to identify new drugs or toxic compounds by their mode of action reflected by the metabolome.

It is to be understood that the analysis of the metabolome shall be carried out after a time period sufficient to allow the compound to enter its effector cells, tissues or organs, i.e. an incubation time period. In other words, the analysis should be carried out after the compound has become bioavailable. Depending on the chemical nature of the compound, the person skilled in the art will know what time period shall be sufficient for bioavailability of said compound. Such a time period could be also defined in a pilot experiment. For example, a compound being of similar or identical chemical nature may be linked to a detectable label. Said labelled compound may then be administered to an animal. The time until the label becomes detectable in the suspected effector cells, tissues or organs will be determined and will serve as incubation time period for the method of the present invention.

The analysis of the metabolome also, preferably, comprises taking a sample from each animal of the animal population which will be further analyzed as described before. Suitable samples include cells, tissues or organs of the animal or body fluids including blood, plasma, serum, urine, spinal liquor. It is well known in the art, how such samples may be taken. Suitable techniques include blood sampling, biopsy, liquor sampling, cell sorting etc. A preferred schedule for sampling is described in the Examples. The sample may be subjected to pretreatments. Such pretreatments include enzymatic digestion of biological material which is not suitable for metabolome analysis, extraction procedures to obtain certain metabolites form the sample, fractioning of the sample, e.g., in order to obtain polar and/or non-polar fractions of metabolites, or derivatisation of the metabolites, e.g., prior to chromatography. Said pre-treatment techniques are well known in the art and can be applied by the person skilled in the art without further ado.

Advantageously, due to providing the animal population in accordance with the present invention reliable comparative analysis of the metabolome can be carried out. The results of said reliable analysis in accordance with the method of the present invention will fasten drug discovery and toxicological assessments. Preferably, databases are created which comprise metabolic data (i.e. data on the qualitative and quantitative composition of metabolomes or metabolic profiles) for a mode of action of a compound or metabolic data which reflect administration of toxic compounds or drugs. The metabolic data of said data bases can be used as references when using the method of the present invention for a screen of further compounds suspected to effect the metabolome. It is envisaged that each screen will yield a more comprehensive view on modes of action. Moreover, by comparison with the said metabolic data (reference data) toxic compounds or therapeutically active compounds can be rapidly identified. Preferably, the method of the present invention is carried out in the high-throughput format.

In a preferred embodiment of the method of the present invention, the animal population is kept for a second time period after administering to the animal population the compounds suspected to effect the metabolome in step b) under the housing conditions described referred to above.

More preferably, analyzing of step c) is carried out at least once during said second time period or at least three times during the second time period, wherein the second and any further analysis is carried out after a period of time which is twice the period of time which passed since the previous analysis. Most preferably, the first analysis is carried out seven days after administration of the compound in step b).

It has been found in the study underlying the present invention that it is advantageous to evaluate the metabolome over a time range from acute up to long-term effects including intermediate time points. Thus, the method of the present invention allows repeated measurements during an investigation. Accordingly, analysing of the animals is preferably carried out at least once, preferably two times or most preferably at least three times during the second time period. The time points for analysis are to be selected as to include the early acute effects as well as the chronic or long-term effects. Suitable time points for the analysis can be determined by the person skilled in the art depending on the animal which is analyzed without further ado. Specifically, if a rodent animal population is used, such as a rat or mouse population, a time course of analysis is preferred in which the second and any further analysis is carried out after a period of time which is twice the period of time which passed since the previous analysis, i.e. if the first analysis is done 7 days after administration of the compound, the second analysis shall be done after 14 days and the third analysis shall be done after 28 days after administration. Preferably, if analysis is done by analyzing a body fluid sample, such as blood, it is preferred that the animals of the animal population will be kept for a fasting period (i.e. withdrawal of food) of about 16 to 20 hours before blood sampling. Moreover, carrying out the analysis during a constant time window allows to compensate for metabolic changes which may be caused for instance by the circadian rhythm of the animals of the animal population. Therefore, it is preferred that blood sampling and analysis is done within and at a constant time window for all analyses. Preferably analysis including sampling is done between 7.30 and 10.30 a.m. Moreover, the methods of the present invention may further encompass in addition to the analysis of the blood metabolome further investigations of the metabolome of different organs or body fluids of the animals of the animal population.

Moreover, in addition to the metabolome investigations, the method may comprise further investigations which do not relate to the metabolome. Specifically, it is envisaged that each animal of the animal population will after the metabolic analysis is completed, be pathologically investigated. Said investigation includes necropsy of the animals including careful examination of the external surface of the body, all orifices, the cranial, thoracic and abdominal cavities and their contents. Moreover, a pathologic analysis of the liver, the kidneys, the adrenals, the testis, the epididymidis, the thymus, the spleen, the brain and the heart shall be carried out as appropriate and as described in Guideline No. 407, loc. cit., for instance. Moreover, the histopathological examination shall be carried out, preferably, as described in the following:

The following tissues should be preserved in the most appropriate fixation medium for both the type of tissue and the intended subsequent histopathological examination: all gross lesions, brain (representative regions including cerebrum, cerebellum and pons), spinal cord, stomach, small and large intestines (including Peyer's patches), liver, kidneys, adrenals, spleen, heart, thymus, thyroid. trachea and lungs (preserved by inflation with fixative and then immersion), gonads, accessory sex organs (e.g. uterus, prostate), urinary bladder, lymph nodes (preferably one lymph node covering the route of administration and another one distant from the route of administration to cover systemic effects), peripheral nerve (sciatic or tibial) preferably in close proximity to the muscle, and a section of bone marrow (or, alternatively, a fresh mounted bone marrow aspirate). The clinical and other findings may suggest the need to examine additional tissues. Also any organs considered likely to be target organs based on the known properties of the test compound should be preserved.

Moreover, it also preferred that haematological and biochemical parameters of the animals of the animal population will be determined. It is preferred that data relating to the pathology and the haematological as well as biochemical parameters will be stored in a suitable database for each of the animals of the animal population as animal-related data. The stored data are preferably used for the evaluation of the metabolic data generated by the analysis of the metabolome and/or for data processing. In a preferred embodiment of the method of the present invention, taking into account this animal-related data allows to avoid false positive or false negative results because animals which shall in comparison to their parallels in an animal population an abnormal pathology, haematology or abnormal biochemical parameters may be excluded from data evaluation during data processing. Moreover, it is further preferred to include animal-related data on body weight, food consumption, drinking liquid consumption and clinical signs as well as possible abnormalities which appear when the animals are kept for the first and second time period as specified above.

Thus, in another preferred embodiment of the method of the present invention, said method further comprising monitoring body weight, food consumption, drinking liquid consumption and clinical signs of the animal population. Said monitoring may be, more preferably, be assisted by automation and the monitored data on body weight, food consumption, drinking liquid consumption and clinical science are collected in a database for each of the animal of the animal population.

Moreover, in light of the foregoing, the method of the present invention is, preferably, comprising monitoring abnormalities for each animal of the animal population. The term “abnormalities” as used herein refers to abnormalities which can be easily detected by maintenance stuff, i.e. abnormalities which do usually not require monitoring by a physician. More preferably, the abnormalities are automatically monitored and the data obtained from said monitoring are stored in a database for each individual of the animal population.

As referred to already above, in a preferred embodiment of the method of the present invention, said analyzing comprises comparing the metabolome of the animal population with a reference.

More preferably, comparing comprises generating a metabolic profile of the metabolome of the animals of the animal population and comparing said profile with a reference. Most preferably, a difference in the metabolic profiles is indicative for a compound which effects the metabolome of an animal. It is to be understood that the reference in this most preferred embodiment is a metabolic profile derived from an untreated animal.

The term “metabolic profile” means that a specific fingerprint is established during analysis for each metabolome of an animal of the animal population. Said metabolic profile may be derived from at least one signal (e.g. a peak in mass spectra) obtained from an animal or a sample thereof by the method of the present invention. More preferably, a metabolic profile is derived from a plurality of such signals. The signals may be obtained from a single metabolite or from a plurality of metabolites. It is to be understood that the primary signals may be further processed by suitable techniques as described above. Preferably, a three-dimensional data set is generated by using at least one time resolved separation technique and at least one mass resolved separation technique. Such a data set could be obtained, e.g., from chromatography coupled mass spectrometry as described above. Such a three-dimensional data set may be analyzed by conventional peak determining algorithms, such as ChemStation or AMDIS, which allow for specific detection of maxima and/or minima in said three-dimensional data set. The maxima and minima thus extracted will be the specific metabolic profile for a certain animal metabolome of an animal population. Moreover, the extracted signals, such as the maxima or minima of the peaks may be further processed into characteristic values as a function of the respective time and mass. It is to be understood that the metabolic profiles generated by the aforementioned techniques consist, preferably, of data sets which are dimensionally reduced and, thus, can be easily compared to each other by statistical tests, including principal component analysis (PCA) or partial least square tests (PLS). A difference in the metabolic profile of a metabolome of a test animal (i.e. an animal which has obtained a compound suspected to effect the metabolome of an animal) and a reference is an indicator that the compound indeed induces metabolic changes in the test animal. However as described already above, this does not necessarily mean that the quantitative composition of the metabolome (i.e. the number of compounds which are present) will be changed. Changes in the metabolic profiles may be also an indicator for an altered quantitative composition of the metabolome (i.e. the amount of the compounds which are present is altered). More preferably, the metabolic profiles may be compared to metabolic profiles comprised by a data base with reference profiles for known drugs, prodrugs or toxic compounds or their modes of action. Advantageously, the method of the present invention, thus, allows to make screening procedures for drugs and drug candidates more time-efficient since toxic or harmful effects of the compounds may be determined at an early stage of drug development. Further, toxicity tests can be easily carried out in a time-efficient manner since the onset of toxic events can be monitored by changes of the metabolic profiles. In particular, this is advantageous for compounds which elicit long-term toxic effects instead of immediate effects.

It is to be understood that differences or changes in the metabolome of an animal can be also analyzed by comparing specific metabolites of known or unknown chemical nature as opposed to a metabolic profile.

Therefore, in another preferred embodiment of the method of the present invention, comparing comprises comparing of at least one metabolite of the metabolome of the animal population with a reference. Most preferably, a difference in said at least one metabolite is indicative for a compound which effects the metabolome of an animal. It is to be understood that the reference in this most preferred embodiment is a metabolic profile derived from an untreated animal.

In another preferred embodiment of the method of the present invention and in light of the foregoing, said compound is a compound suspected to be toxic or a compound suspected to be a drug.

The present invention further relates to a method for the identification of a marker for a compound which effects the metabolome of an animal, comprising the steps of the method of the present invention as described before and the further step of providing a marker for said compound based on the analysis of the metabolome. In a preferred embodiment of the method of the present invention, said marker is a metabolic profile of the animal population which is altered compared to a reference. Most preferably, said marker indicates toxicity of a compound, a mode of action of a compound or a therapeutic activity of a compound.

Moreover, the present invention encompasses a method for identifying a compound which effects the metabolome of an animal comprising metabolically analyzing a sample from at least one animal of an animal population to which a compound suspected to effect the metabolome has been administered and wherein said animal population has been kept prior and after administration of the compound under the housing conditions referred to before.

The term “metabolically analyzing” as used herein, preferably, encompasses all means, methods and embodiments described before for analyzing the metabolome of an animal.

The term “sample” as used herein encompasses samples of biological material obtained from the animal to be investigated. Suitable sources for samples have been described above already.

In a preferred embodiment of the method of the present invention, said analyzing comprises comparing the metabolome from the sample of an animal of said animal population with a reference.

More preferably, comparing comprises generating a metabolic profile for the sample of an animal of the animal population and comparing said profile with a reference. Most preferably, a difference in the metabolic profile is indicative for a compound which effects the metabolome of an animal. It is to be understood that the reference in this preferred embodiment is a metabolic profile derived from an untreated animal.

Also more preferably, comparing comprises comparing at least one metabolite of the metabolome from the sample of an animal of the animal population with a reference. Most preferably, a difference in the said at least one metabolite is indicative for a compound which effects the metabolome of an animal. It is to be understood that the reference in this most preferred embodiment is a metabolic profile derived from an untreated animal.

Also encompassed by the present invention is a method for the identification of a marker for a compound which effects the metabolome of an animal comprising the steps of the aforementioned methods (based on samples) and the further step of providing said marker based on the analysis of the metabolome.

In a preferred embodiment of said method of the present invention, said marker is a metabolic profile or is at least one metabolite from the metabolome of the sample of an animal of the animal population which is altered compared to a reference. Most preferably, said marker indicates toxicity of a compound, a mode of action of a compound or a therapeutic activity of a compound.

The invention will be now illustrated by the following Examples. However, the Examples are merely for purpose of illustration and not meant to limit the scope of the invention.

### EXAMPLE 1

### Animal Keeping

Rats of the strain CrlGlxBrlHan:Wi were purchased from Charles River, Sulzfeld, Germany having an age of 63 to 65 days. Each animal has been labelled by an ear tattoo, consecutively. Animals were kept under the following housing conditions:

| | |
|---|---|
| Air conditions: | Temperature 20-24° C., humidity 30-70%. |
| | Any deviations have been documented. |
| Illumination | 12 hours light from 6.00 to 18.00 hours, 12 hours |
| period: | darkness from 18.00 to 6.00 hours |
| Type of cage: | Wire cages, type DK III, BECKER & Co., Castrop- |
| | Rauxel, Germany |
| No. of animals | 1 |
| per cage: | |
| Type of diet: | Ground Kliba mouse/rat maintenance diet “GLP”, |
| | meal, supplied by Provimi Kliba SA, Kaiseraugst |
| | (Switzerland), ad libitum |
| Watering: | Drinking water ad libitum |
| Acclimatization: | During the 7 day acclimatization period, the |
| | animals have been accustomed to the environmental |
| | conditions of the study and to the diet. |

### EXAMPLE 2

### Metabolic Investigation of Test Compounds

Male and female wistar rats have been randomized and allocated to the dose groups before the beginning of the administration period on the basis of their weights. The animals have been treated with five different test compounds at a high and low dose level according to the following schedule shown in Table 1.

TABLE 1

| | | | | |
|---|---|---|---|---|
| | | Dose level | No. of | |
| Dose | Test | (ppm | animals | Animal no. |

| group | substance | in the diet) | per sex | males | Females |
|---|---|---|---|---|---|
| 00 | 0 | 0 | 10 | 1-10 | 61-70 |
| 01 | A | Low dose | 5 | 11-15 | 71-75 |
| 02 | A | High dose | 5 | 16-20 | 76-80 |
| 03 | B | Low dose | 5 | 21-25 | 81-85 |
| 04 | B | High dose | 5 | 26-30 | 86-90 |
| 05 | C | Low dose | 5 | 31-35 | 91-95 |
| 06 | C | High dose | 5 | 36-40 | 96-100 |
| 07 | D | Low dose | 5 | 41-45 | 101-105 |
| 08 | D | High dose | 5 | 46-50 | 106-110 |
| 09 | E | Low dose | 5 | 51-55 | 11-115 |
| 10 | E | High dose | 5 | 56-60 | 116-120 |

Blood sampling was carried out as indicated in the following time schedule shown in Table 2.

TABLE 2

| Date | Phase of study/Examination | Date of study |
|---|---|---|
| | Experimental starting date: Arrival of the | −6 |
| | animals and start of acclimatization period | |
| | Randomization of the animals | |
| | Start of administration period | 0 |
| | Blood sampling¹⁾²⁾ | 7 |
| | Blood sampling¹⁾²⁾ | 14 |
| | Blood sampling¹⁾²⁾ and necropsy | 28 |

Blood sample preparation
Evaluation of the clinical findings
Summary of the clinical results

¹⁾= Before necropsy/blood examination fasting period (withdrawal of food) of about 16 to 20 hours
²⁾= Between 07:30 and 10:30

During the experiment, a check for moribund and dead animals has been made twice, daily from Monday to Friday and once daily on Saturday, Sunday and public holidays. The animals will be checked daily for any clinical abnormal signs. Abnormalities and changes will be documented for each animal. The food consumption has been determined on study days 6, 13, 20 and 27. Drinking water consumption has been checked daily within the general observations. Body weight has been determined before the start of the administration period, in order to randomize the animals. During the administration period the body weight has been determined on study days 0, 6, 13, 20 and 27. The mean daily intake of the test substances have been calculated based upon individual values for body weight and food consumption. Means and standard deviations have been calculated using Dunnet's test.

Blood sampling was carried out as follows:

Before necropsy or blood sampling, food was withdrawn for about 16 to 20 hours (fasting period). Blood sampling was done between 7:30 and 10:30 a.m. Blood was taken from the retroorbital venous plexus of isoflurane anaesthesized animals. From each animal 1 ml of blood will be collected in plastic tubes with EDTA as anticoagulant (10 μl of a 10% solution). The blood samples are centrifuged. The plasma of each sample is separated and transferred to another plastic tube. The precipitated erythrocytes are washed three times with 0.9% NaCl, and filled up ad 1 ml with sterile distilled water (Ampuwa®, Fresenius, Bad Homburg, Germany) in order to hemolyse red blood cells. Hemoglobin is determined in the hemolysed blood samples (40 μl hemolysate plus 160 μl 1.5% NaCl) with an automatic analyzer (ADVIA 120, Bayer AG, Femwald, Germany). Blood, plasma and hemolysate are sampled and stored in original Eppendorf tubes. Transport and preparation of the samples are done under cooling with ice. At the end of sample preparation all samples are overlaid with an atmosphere of pure nitrogen, sealed with “Parafilm M” and stored at −80° C. (under nitrogen atmosphere) until further prosecution of the samples (e.g., shipment).

After completion of the experiment, clinical pathology for each animal was determined. To this end all animals which survived the study have been sacrificed by decapitation under isoflorane anaesthesia (if final blood sampling was envisaged) or by CO₂ anaesthesia.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method for providing a mammalian animal population, the members of which have an essentially identical metabolome, the method comprising:
a) compiling a mammalian animal population, the members of which are essentially of the same age;
b) maintaining the population of step a) for a first time period sufficient for acclimatization under the housing conditions of:
i) constant temperature;
ii) constant humidity;
iii) physical separation of the mammals of the mammalian population;
iv) feeding ad libitum, wherein the food to be fed is essentially free of chemical or microbial contaminants;
v) drinking ad libitum, wherein the drinking liquid is essentially free of chemical or microbial contaminants;
vi) constant illumination period; and
c) providing the mammalian animal population of step b) after the first time period.

32. The method of claim 31 wherein the mammalian animal is a rodent.

33. The method of claim 32 wherein the rodent is a rat or a mouse.

34. The method of claim 33 wherein the age range of the rat or mouse is plus or minus 1 day of a target age of the population.

35. A method for the identification of a compound that affects the metabolome of a mammal comprising:
a) compiling a mammalian animal population, the members of which are essentially of the same age;
b) maintaining the population of step a) for a first time period sufficient for acclimatization under the housing conditions of:
i) constant temperature;
ii) constant humidity;
iii) physical separation of the mammals of the mammalian population;
iv) feeding ad libitum, wherein the food to be fed is essentially free of chemical or microbial contaminants;
v) drinking ad libitum, wherein the drinking liquid is essentially free of chemical or microbial contaminants;
vi) constant illumination period;
c) providing the mammalian animal population of step b) after the first time period;
d) administering to the population a compound suspected of affecting the metabolome of a mammal; and
e) then analyzing the metabolome of the population.

36. The method of claim 35 wherein step b) further comprises maintaining the population under the housing conditions of claim 31 for a second time period after administering the compound.

37. The method of claim 36 wherein analysis of the metabolome of the population is carried out at least once during the second time period.

38. The method of claim 36 wherein analysis of the metabolome of the population is carried out at least three times during the second time period, and wherein the second and each further analysis is carried out after a time period that is twice the time period since the previous analysis.

39. The method of claim 38 wherein the first analysis is carried out seven days after administration of a compound suspected to affect the metabolome of a mammal.

40. The method of claim 35 further comprising monitoring body weight, food consumption, drinking liquid consumption and clinical signs of the mammalian population.

41. The method of claim 35 wherein analysis comprises comparing the metabolome of the mammalian animal population with a reference.

42. A method for the identification of a marker for a compound that affects the metabolome of a mammal comprising:
a) compiling a mammalian animal population, the members of which are essentially of the same age;
b) maintaining the population of step a) for a first time period sufficient for acclimatization under the housing conditions of:
i) constant temperature;
ii) constant humidity;
iii) physical separation of the mammals of the mammalian population;
iv) feeding ad libitum, wherein the food to be fed is essentially free of chemical or microbial contaminants;
v) drinking ad libitum, wherein the drinking liquid is essentially free of chemical or microbial contaminants;
vi) constant illumination period;
c) providing the mammalian animal population of step b) after the first time period;
d) administering to the population a compound suspected of affecting the metabolome of a mammal;
e) then analyzing the metabolome of the population; and
f) providing a marker for the compound based on the analysis of the metabolome.

43. The method of claim 42 wherein the marker is a metabolic profile or is at least one metabolite of the mammalian animal population that is altered relative to a reference.

44. The method of claim 42 wherein the marker indicates toxicity of a compound, a mode of action of a compound or a therapeutic activity of a compound.
